(19) United States
(12) Patent Application Publication
(10) Pub. No.: US 2020/0185107 A1
(43) Pub. Date: Jun. 11, 2020

(54) DIGITAL TWIN OPERATION

(71) Applicant: KONINKLIJKE PHILIPS N.V., Eindhoven (NL)

(72) Inventors: LIEKE GERTRUDA ELISABETH COX, EINDHOVEN (NL); CORNELIS PETRUS HENDRIKS, EINDHOVEN (NL); MURTAZA BULUT, EINDHOVEN (NL); VALENTINA LAVEZZO, EINDHOVEN (NL); OLAF VAN DER SLUIS, LANDGRAAF (NL)

(21) Appl. No.: 16/704,495

(22) Filed: Dec. 5, 2019

Related U.S. Application Data
(63) Continuation of application No. 62/775,443, filed on Dec. 5, 2018.

(51) Int. Cl. G16H 50/50; G06F 30/20; A61B 5/00

(52) U.S. Cl. CPC G16H 50/50; A61B 5/0022; G06F 30/20

(57) ABSTRACT

Disclosed is a computer system (20) comprising a processor arrangement (22) communicatively coupled to a data storage arrangement (30) storing a virtual model of a patient, said virtual model comprising at least one of a digital representation of at least a part of the anatomy of the patient and a physiological model of a bodily process of the patient; and a communication module (24) communicatively coupled to said processor arrangement and arranged to receive sensor data from one or more sensors (12, 14, 16) arranged to monitor said patient, wherein the processor arrangement is arranged to retrieve (103) said virtual model from the data storage arrangement; receive (105) said sensor data from the communication module; evaluate said sensor data with said virtual model; generate (111, 121) an instruction for altering a mode of operation of at least one sensor of the one or more sensors in response to said evaluation or in response to a user request; and transmit said instruction to the at least one sensor or to a device for invoking control of said at least one sensor with the communication module. Also disclosed is a method for operating a computer system in such a manner and a computer program product for implementing such a method.

### FIELD OF THE INVENTION

The present invention relates to a computer system comprising a processor arrangement communicatively coupled to a data storage arrangement storing a virtual model of a patient, said virtual model comprising a digital representation of at least a part of said patient; and a communication module communicatively coupled to said processor arrangement.

The present invention further relates to a method of controlling the one or more sensors monitoring such a patient with such a computer system.

The present invention further relates to a computer program product for implementing such a method with such a computer system.

### BACKGROUND OF THE INVENTION

In many developed countries, the provision of healthcare is becoming increasingly strained. Some reasons for this include the growth of the population and increasing life expectancy. Unfortunately, although people live longer, the average age at which their health deteriorates to the point where regular medical care is required is not increasing accordingly, such that the ageing population is unwell for longer, which increases the pressure on the healthcare system, e.g. on medical practitioners, medical infrastructures such as hospitals, diagnostic equipment therein, and so on. Hence, rather than simply increasing medical resources, for which the financial resources may not be available, there exists a need to improve the efficiency of such healthcare systems.

A recent development in healthcare is the so-called digital twin concept. In this concept, a digital representation (the digital twin) of a physical system is provided and connected to its physical counterpart, for example through the Internet of things as explained in US 2017/286572 A1 for example. Through this connection, the digital twin typically receives data pertaining to the state of the physical system, such as sensor readings or the like, based on which the digital twin can predict the actual or future status of the physical system, e.g. through simulation. In case of electromechanical systems, this for example may be used to predict the end-of-life of components of the system, thereby reducing the risk of component failure as timely replacement of the component may be arranged based on its end-of-life as estimated by the digital twin.

Such digital twin technology is also becoming of interest in the medical field, as it provides an approach to more efficient medical care provision. For example, the digital twin may be built using imaging data of the patient, e.g. a patient suffering from a diagnosed medical condition as captured in the imaging data, as for instance is explained by Dr Vanessa Diaz in https://www.wareable.com/health-and-wellbeing/doctor-virtual-twin-digital-patient-ucl-887 as retrieved from the Internet on 29 Oct. 2018. Such a digital twin may serve a number of purposes. Firstly, the digital twin rather than the patient may be subjected to a number of virtual tests, e.g. treatment plans, to determine which treatment plan is most likely to be successful to the patient. This therefore reduces the number of tests that physically need to be performed on the actual patient.

The digital twin of the patient for instance further may be used to predict the onset, treatment (outcome) or development of such medical conditions of the patient using the patient-derived digital model. To this end, the patient may be fitted with one or more sensors that are connected to the digital twin. The digital twin typically uses sensor readings provided by the one or more sensors to assess the actual medical status of the patient, for example by developing the patient-specific model using the received sensor readings.

In this manner, the medical status of a patient may be monitored without the routine involvement of a medical practitioner, e.g. thus avoiding periodic routine physical checks of the patient. Instead, only when the digital twin predicts a medical status of the patient indicative of the patient requiring medical attention based on the received sensor readings may the digital twin arrange for an appointment to see a medical practitioner to be made for the patient. This typically leads to a reduction in such appointments, thereby freeing up the medical practitioner to see other patients. Moreover, major medical incidents that the patient may be about to suffer may be predicted by the digital twin based on the monitoring of the patient's sensor readings, thereby reducing the risk of such incidents actually occurring. Such prevention avoids the need for the provision of substantial aftercare following such a major medical incident, which also alleviates the pressure on a healthcare system otherwise providing such aftercare.

Such remote monitoring of a patient may lead to an infrequent need for the patient to physically meet a healthcare professional. Consequently, any sensors that are used to monitor such a patient should have a battery life that enables the sensors to operate for an as long as possible continuous period of time, e.g. to ensure that reliable data acquisition during such a monitoring period is safeguarded, without the patient or healthcare professional having to recharge or replace the batteries of such sensors, as such recharging or replacing not only can be cumbersome but can also lead to gaps in the monitoring data. More generally speaking, it is desirable that the one or more sensors monitoring the patient are operated such that inconvenience or discomfort to the patient is minimized or at least reduced.

### SUMMARY OF THE INVENTION

The present invention seeks to provide a computer system implementing a digital representation of at least a part of a patient that is configured to extend the operational life of the one or more sensors that monitor the physical entity.

The present invention further seeks to provide a method to control the one or more sensors monitoring such a patient with such a computer system in order to extend the operational life of the one or more sensors.

The present invention further seeks to provide a computer program product for implementing such a method on such a computer system.

According to an aspect, there is provided a computer system comprising a processor arrangement communicatively coupled to a data storage arrangement storing a virtual model of a patient, said virtual model comprising at least one of a digital representation of at least a part of the anatomy of the patient and a physiological model of a bodily process of the patient; and a communication module communicatively coupled to said processor arrangement and arranged to receive sensor data from one or more sensors arranged to monitor said patient, wherein the processor arrangement is arranged to retrieve said virtual model from the data storage arrangement; receive said sensor data from the communication module; evaluate said sensor data with said virtual model; generate an instruction for altering a mode of operation of at least one sensor of the one or more sensors in response to said evaluation or in response to a user request; and transmit said instruction to the at least one sensor or to a device for invoking control of said at least one sensor with the communication module.

The present invention is based on the realization that the digital twin of the patient as implemented by the computer system, i.e. the digital representation of at least a part of the anatomy or physiology of the patient, may be used to control certain modes of operation of the one or more sensors such as a sampling or operating frequency of such sensors in order to reduce inconvenience to the patient, for example by preserving operational life of the one or more sensors such that the patient does not have to frequently replace or recharge sensor batteries, or by limiting discomfort to the patient by only invoking certain types of sensor measurements, e.g. blood pressure measurements, that may be perceived as uncomfortable by the patient. More specifically, the digital representation is used in the evaluation to decide whether there is a need to change a mode of operation of a sensor monitoring the patient. In case the virtual model comprises a physiological model of the patient, in some embodiments this model may be a disease model, in which case the sensor data may facilitate an estimation of an actual or future stage of the disease suffered by the patient with the virtual model by virtue of monitoring physiological parameters of the patient's body that are relevant to such a physiological model. In this context, such physiological parameters may include vital signs, such as heart rate, blood pressure, breathing rate, body temperature, and substance levels in bodily fluids such as blood glucose level, blood insulin level, and so on. In other words, such physiological parameters may be any parameter from which the functioning of (part of) the patient's body or physiology may be derived. Alternatively, rather than the instruction being generated based on the evaluation of the sensor data, the computer system may receive a user request, e.g. through a user interface communicatively coupled to the computer system, for changing a mode of operation of at least one sensor of the one or more sensors monitoring the patient. For example, a healthcare professional may seek to simulate different treatment options with the digital twin for a medical condition the patient is suffering from, which simulation may require additional, more up-to-date and/or more accurate sensor data in order for the simulation to have the desired degree of accuracy.

In a first set of embodiments, the evaluation of the received sensor data is used to obtain an indication of the actual or future health (physical condition) of the patient. To this end, the computer system may be arranged to evaluate said sensor data with said virtual model by simulating an actual or future physical condition of said patient by developing said digital representation based on the received sensor data and generate said instruction based on said simulated actual or future physical condition. In this manner, the one or more sensors monitoring the patient may be operated in an energy saving mode as long as the patient's physical condition is stable, and may be switched to a more energy consuming mode, e.g. a higher sampling frequency, when the simulation indicates that the equilibrium of the patient's physical condition is likely to be disturbed and a closer monitoring of the patient becomes desirable. Such a change in sensor settings for example may be invoked if the digital twin predicts a relevant change between a simulated actual or future physical condition of the patient and at least one previous physical condition of the patient, e.g. a change between two simulated physical conditions simulating the physical condition of the patient at different points in time, a simulated physical condition exceeding a defined threshold, a trend in a series of simulated physical conditions representing the physical condition of the patient at different points in time indicating a worrying change in the patient's physical condition and so on. It is noted that the original or starting mode of operation of the one or more sensors may be set in any suitable manner.

The processor arrangement may be arranged to simulate the actual or future physical condition of said patient by developing said digital representation based on the received sensor data and received user information indicative of said actual physical condition. Such user information, which may be provided using a user interface of one of said sensors or alternatively which may be provided through a user interface of the computer system communicatively coupled to the processor arrangement for providing said user information, may supplement the sensor data, thereby enabling the computer system to generate a more accurate simulation of the patient's actual or future physical condition to a more complete set of data being made available for said simulation. For example, such user input may include user-reported symptoms that may be indicative of an actual or future disturbance of the equilibrium of the physical condition of the patient.

In an embodiment, the one or more sensors include a first sensor arranged to monitor a first physiological parameter of the patient that is related to a second physiological parameter of the patient and a second sensor arranged to monitor said second physiological parameter, and wherein the processor arrangement is arranged to simulate the actual or future physical condition from sensor data provided by the first sensor; verify the simulated actual or future physical condition from sensor data provided by the second sensor; and generate the instruction if said simulated actual or future physical condition has successfully been verified and is indicative of a relevant change in the physical condition of the patient. This for example is particularly useful where monitoring the second physiological parameter related to the physical condition of interest is rather energy demanding, such that it is more energy efficient to monitor the first physiological parameter that is linked or related to the second physiological parameter, i.e. to the same physical condition to which the second physiological parameter is related.

For example, based on the simulated actual or future physical condition of the patient, the processor arrangement may be further arranged to generate a further instruction for activating the second sensor and transmitting the further instruction to the second sensor with the communication module based on the simulated actual or future physical condition such that the second sensor is only used to verify a change in the actual or future physical condition as predicted from the sensor data of the first sensor. This for example may be useful where the second operating parameter provides a more accurate monitoring of a patient's physical condition when in flux.

In another embodiment, wherein the one or more sensors include a first sensor arranged to monitor a first physiological parameter of the patient that is related to a second physiological parameter of the patient and a second sensor arranged to monitor said second physiological parameter, and wherein the processor arrangement is arranged to generate a further instruction for activating the second sensor and transmitting the further instruction to the second sensor with the communication module based on said simulated actual or future physical condition. This for instance is useful where the sensor data of the first sensor indicates a relevant change in the patient's actual physical condition, such that one or more additional sensors monitoring physiological parameters of the patient that are related to the patient's actual physical condition may be activated in order to more closely monitor the actual physical condition, such as to more accurately monitor further changes in the actual physical condition that may lead to a critical event without timely intervention. For example, the actual physical condition may be a heart condition of the patient as monitored by a heart rate sensor attached to the patient, in which case a relevant change in the heart condition as suggested by a change in the heart rate may trigger the activation of a blood pressure sensor and/or a respiration rate sensor attached to the patient to more holistically monitor further changes to the heart condition, as heart rate alone may not provide a complete insight into such further changes to the heart condition of the patient.

In a second set of embodiments, the computer system does not necessarily simulate an actual physical condition of the patient with the virtual model. In these embodiments (which may be combined with the first set of embodiments), the processor arrangement is arranged to evaluate said sensor data with said virtual model by determining a quality indicator of said sensor data and generate said instruction if said quality indicator is below a defined threshold. Such a quality indicator for example may be an indicator of the relevance of the sensor data, an indicator of a signal quality of the sensor data, and so on. In this embodiment, the virtual model is used to assess the reliability and/or relevance of the sensor data without developing a higher level simulated physical condition of the patient with the virtual model using the sensor data. This for instance has the advantage that meaningless simulations due to unreliable or outdated sensor data are avoided.

Alternatively or additionally, the processor arrangement may be arranged to evaluate said sensor data with said virtual model by determining or receiving an operational life indication of the one or more sensors with said sensor data and generating said instruction in response to said operational life indication. In this manner, the digital twin may decide whether it is (clinically) acceptable to alter a mode of operation of a sensor to prolong its operational life. In the context of the present application, the operational life of a sensor may refer to its remaining battery charge as well as to the estimated lifetime of such a sensor or a component thereof, which for example may be estimated from mechanical, thermal, chemical characteristics and so on associated with the operation of such a sensor or component.

Such resource evaluation in order to control the mode of operation at least some of the sensors monitoring the patient is not limited to the evaluation of the operational state of the sensors. For example, the processor arrangement may be arranged to determine a remaining capacity of a data storage arrangement used by the virtual model to store the sensor data and generate said instruction in response to said determined remaining capacity. In this manner, the digital twin may decide whether it is (clinically) acceptable to reduce a data volume produced by the one or more sensors to avoid the data storage arrangement from becoming fully utilized. Such a data storage arrangement may take any suitable form, such as a hard disk, solid state disk, memory, cache, and so on. In addition, this embodiment may further cover computational and processing capacity of the computer system.

The processor arrangement may be further arranged to adjust said instruction and transmit said adjusted instruction to the at least one sensor with the communication module in response to an indication of an inability to comply with the original instruction from the at least one sensor. For example, such an original instruction may comprise an instruction to operate a sensor in a particular mode of operation for a particular duration. The controller of such a sensor may determine whether the sensor is capable of performing this mode of operation for the full duration, e.g. in terms of remaining battery life. Where this is not the case, the sensor (e.g. its controller) may notify the computer system of its inability to fully comply with the original instruction, upon which the processor arrangement may adjust and reissue the instruction accordingly, e.g. based on battery life information included in the notification as provided by the sensor (controller).

According to another aspect, there is provided a method of controlling one or more sensors arranged to monitor a patient with a computer system comprising a processor arrangement communicatively coupled to a data storage arrangement storing a virtual model of the patient, said virtual model comprising at least one of a digital representation of at least a part of the anatomy of the patient and a physiological model of a bodily process of the patient; and a communication module communicatively coupled to said processor arrangement and arranged to receive sensor data from one or more sensors arranged to monitor said patient, the method comprising, with said processor arrangement, retrieving said virtual model from the data storage arrangement; receiving said sensor data from the communication module; evaluating said sensor data with said virtual model; generating an instruction for altering a mode of operation of at least one sensor of the one or more sensors in response to said evaluation or in response to a user request; and transmitting said instruction to at least one sensor or to a device for invoking control of said at least one sensor with the communication module. With such a method, the sensors can be operated in a particularly energy efficient manner under control of the virtual model, or more generally speaking, inconvenience or discomfort to the patient can be reduced or minimized as explained in more detail above.

In a first set of embodiments, evaluating said sensor data with said virtual model comprises simulating an actual or future physical condition of said patient by developing said digital representation based on the received sensor data; and generating said instruction based on the simulated actual or future physical condition. This ensures that the one or more sensors are only operated in a higher energy consuming mode of operation if the virtual model for instance suggests a medical need for such operation, as explained in more detail above.

In an embodiment, the one or more sensors include a first sensor arranged to monitor a first physiological parameter of the patient that is related to a second physiological parameter of the patient and a second sensor arranged to monitor said second operating parameter, and wherein the method comprises simulating the actual or future physical condition from sensor data provided by the first sensor, verifying the simulated actual or future physical condition from sensor data provided by the second sensor; and generating the instruction based on the simulated actual or future physical condition if said simulated actual or future physical condition has successfully been verified. In this manner, the actual physical condition may be monitored by a minimal number of sensors as long as the patient's actual physical condition is in equilibrium, i.e. is stable, with one or more additional sensors only being used to verify changes to the actual physical condition when this condition for instance is suspected to be in flux, thereby providing added accuracy to the prediction of the changes to the physical condition of the patient.

The method optionally further comprises generating a further instruction for activating the second sensor with the processor arrangement and transmitting the further instruction to the second sensor with the communication module based on the simulated actual or future physical condition such that the one or more additional sensors to verify the simulated change in the patient's physical condition are only activated when such verification is required, thereby further improving the energy efficiency of the sensor arrangement monitoring respective physiological parameters of the patient.

In another embodiment, the one or more sensors include a first sensor arranged to monitor a first physiological parameter of the patient that is related to a second physiological parameter of the patient and a second sensor arranged to monitor said second physiological parameter; and the method further comprising generating a further instruction for activating the second sensor with the processor arrangement and transmitting the further instruction to the second sensor with the communication module based on the simulated actual or future physical condition. In this embodiment, additional sensors may be only activated when the patient's physical condition is suspected to be in flux, e.g. in order to more accurately monitor changes to this physical condition by monitoring more operating parameters pertaining to this physical condition, which again provides a particularly energy-efficient implementation of the sensor arrangement monitoring respective physiological parameters of the patient.

The method may further comprise evaluating said sensor data with said virtual model by determining a quality indicator of said sensor data and generating said instruction if said quality indicator is below a defined threshold. As explained above, in this manner the reliability and/or relevance of the sensor data may be assessed without the need to generate a high-level simulation of the actual physical condition of the patient. Rather, the operation of the one or more sensors may be adjusted using the virtual model using such low-level criteria.

The method may further comprise receiving a battery life indication of the one or more sensors with said sensor data and generating said instruction in response to said battery life indication, e.g. to prolong the battery life of such sensors.

The method may further comprise determining a remaining capacity of a data storage arrangement used by the virtual model to store the sensor data and generating said instruction in response to said determined remaining capacity, e.g. to avoid or delay such a data storage arrangement from becoming fully utilized.

The method may further comprise adjusting said instruction with the processor arrangement and transmitting said adjusted instruction to the at least one sensor with the communication module in response to an indication of an inability to comply with the original instruction from the at least one sensor. In this manner, the method may factor in an actual state of the sensor for which the original instruction was intended, thereby further safeguarding the operational lifetime of such a sensor, e.g. a battery-powered sensor.

According to yet another aspect, there is provided a computer program product for a computer system comprising a processor arrangement communicatively coupled to a data storage arrangement storing a virtual model of a patient, said virtual model comprising at least one of a digital representation of at least a part of the anatomy of the patient and a physiological model of a bodily process of the patient; and a communication module communicatively coupled to said processor arrangement and arranged to receive sensor data from said one or more sensors; the computer program product comprising a computer readable storage medium having computer readable program instructions embodied therewith for, when executed on the processor arrangement, cause the processor arrangement to implement the method of any of the herein described embodiments. Such a computer program product for instance may be used to configure existing computer systems to implement the method according to embodiments of the present invention.

### BRIEF DESCRIPTION OF THE DRAWINGS

Embodiments of the invention are described in more detail and by way of non-limiting examples with reference to the accompanying drawings, wherein:

FIG. 1 schematically depicts a patient sensor arrangement coupled to a computer system according to an example embodiment;

FIG. 2 depicts a flowchart of a method according to an embodiment;

FIG. 3 depicts a flowchart of a method according to another embodiment;

FIG. 4 depicts a flowchart of a method according to yet another embodiment; and

FIG. 5 depicts a flowchart of a method according to yet another embodiment.

### DETAILED DESCRIPTION OF THE EMBODIMENTS

It should be understood that the Figures are merely schematic and are not drawn to scale. It should also be understood that the same reference numerals are used throughout the Figures to indicate the same or similar parts.

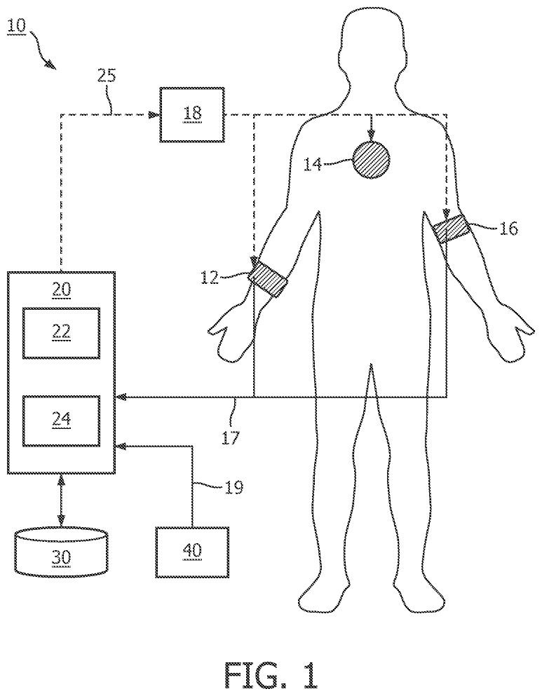

FIG. 1 schematically depicts a generalised setup to which embodiments of the present invention are applicable. A patient 10 is monitored by one or more sensors, here schematically depicted by sensors 12, 14 and 16 by way of non-limiting example only, which one or more sensors are arranged to provide sensor data to a computer system 20 comprising a processor arrangement 22 and a data communication module 24 to which the one or more sensors are communicatively coupled through a data link 17. The processor arrangement 22 of the computer system 20 may take any suitable shape. In the context of the present invention, a processor arrangement may comprise one or more processors, processor cores and the like that cooperate to form such a processor arrangement. Similarly, the data communication module 24 may take any suitable shape, such as a wireless or wired data communication module, as is well known in the art and will therefore not be further explained for the sake of brevity only.

The data link 17 may take any suitable shape, such as a wireless communication link, a wired communication link or a combination thereof. Any suitable communication protocol may be deployed between the one or more sensors and the communication module 24 over the data link 17. For example, in case of a wireless communication link, the communication protocol may be Wi-Fi, Bluetooth, a mobile phone communication protocol such as 3G, 4G, 5G and so on. Other examples of suitable wireless communication links will be immediately apparent to the skilled person. In case of a wired communication link, suitable application protocols may include TCP/IP and similar protocols used to communicate over a wired data communication link such as a wired network, e.g. the Internet.

The computer system 20 is typically remote from the patient 10 such that the patient 10 may be monitored from a distance. For example, the computer system 20 may comprise a remote server or the like on which the digital twin of the patient 10 is hosted.

The one or more sensors typically are each arranged to monitor a physiological parameter of the patient 10. Such parameters are typically indicative of a physical condition of the patient 10. The one or more sensors 12, 14, 16 may be wearable sensors, e.g. battery-powered wearable sensors, epidermal sensors and/or may be sensors implanted into the body of the patient, which typically are also battery powered sensors. Moreover, such sensors do not need to be in physical contact with the patient. For example, sensors in the environment of the patient or in electronic devices used by the patient may also monitor such physiological parameters. Such sensors 12, 14, 16 may electrically, mechanically, thermally, chemically or optically measure digital signal and parameters of the patient 10 from which physiological indicators such as temperature, heart rate, blood pressure, blood flow rate, fractional flow reserve, respiration rate, blood chemistry such as blood glucose level, sweat levels, brain activity (EEG), motion, speech, image-based monitoring (e.g. to monitor body regions of the patient) and so on can be calculated or estimated. As such sensors 12, 14, 16 may be battery-powered sensors having a finite operational lifetime as defined by the battery charge, it is desirable to extend the operational lifetime as much as possible as will be explained in further detail below.

The one or more sensors 12, 14, 16 may each include at least one of a photoplethysmography (PPG) sensor and an accelerometry sensor to determine physiological parameters such as heart rate, activity level and type, e.g. the number of steps taken by the patient 10 over a certain period of time, energy expenditure, sleep parameters and so on, for example during an active period of the patient 10. Other parameters, such as respiration rate and blood pressure changes may be monitored with such sensors when the patient 10 is at rest. Other physiological parameters that may be monitored with such or other sensors include blood glucose levels, bladder fill levels, blood flow rates, e.g. using Doppler ultrasound sensors, and so on. The skilled person will understand that the teachings of the present application are not limited to a particular type of sensor and may be any type of sensor that can be used to monitor a physiological parameter of the patient 10.

The computer system 20 may be communicatively coupled to a data storage device 30, which may store a digital model of the patient 10. Any suitable type of data storage device 30 may be used for this purpose, such as a data storage device 30 forming part of the computer system 20, or a data storage device 30 that is accessible by the computer system 20 over a network such as a storage area network (SAN) device, a network attached storage (NAS) device, a cloud storage device, and so on. Such a digital model in the remainder of this application will also be referred to as a digital twin of the patient 10. The digital twin hosted by the computer system 20 typically provides a biophysical model that is specific to the patient, and typically simulates at least a part of the patient's anatomy, such as (part of) the patient's cardiovascular system, the patient's pulmonary system, the patient's digestive system, a metabolic process of the patient, and so on. Such a biophysical model may be developed from patient data, e.g. imaging data such as CT images, MRI images, ultrasound images, and so on. A typical workflow for creating and validating a 3D, subject-specific biophysical model is depicted in “Current progress in patient-specific modeling”, by Neal and Kerckhoff, 1, 2009, Vol. 2, pp. 111-126.

Such a remote computer system 20 may be located or accessible in a health care environment such as a surgery, hospital or the like, from which a medical practitioner can remotely monitor the physical state of the patient. Alternatively, such monitoring may be performed automatically such that a consult or procedure for the patient is only scheduled when his or her digital twin predicts the imminent occurrence of a critical medical condition or any other change in the physical condition of the patient that ideally requires the patient to be brought face to face with a health care professional.

The sensor data provided by the one or more sensors 12, 14, 16 to the computer system 20 hosting the digital twin through its processor arrangement 22 is used to update and change the digital twin such that any changes to the patient 10 as highlighted by the sensor data are reflected in the digital twin. As such, the digital twin forms a learning system that learns from itself using the sensor data provided by the one or more sensors 12, 14, 16. The one or more sensors 12, 14, 16 may provide the sensor data based on which the digital twin as executed by the processor arrangement 22 may simulate an actual or future physical condition of the patient by developing the digital twin in order to closely mimic the physical condition of the patient. In this context, the physical condition of the patient may be a health condition or the like. Such an actual physical condition may be simulated for a number of reasons, e.g. to provide insight in the actual physical condition of the patient and/or to validate the digital twin. For example, based on a previously received set of sensor data, the digital twin may have simulated a future physical condition of the patient 10, in which such sensor data may have been used to parameterize the digital twin to facilitate the prediction of such a future physical condition of the patient 10. The actual sensor data may be used to validate such a prediction, e.g. by using the actual sensor data to simulate the physical condition of the patient 10 at the same point in time, e.g. a point in time in the future or the actual point in time, and may be used to update the digital twin if necessary, e.g. if such a validation highlights a discrepancy between the simulated physical conditions using the ‘old’ and ‘new’ sensor data respectively.

Optionally, the digital twin may further consider user information provided by the patient 10 or by a person monitoring the patient 10 to further aid the clinical decision making process. Such information may be information of intermediate diagnostic relevance, such as symptoms, test results, up to date patient image data on which the digital twin of the patient 10 is based, and so on. To this end, the computer system 20 may be communicatively coupled to a user interface 40, which may provide such user information to the computer system 20 over a data communication link 19. The user interface 40 may form part of a sensor of the one or more sensors 12, 14, 16 monitoring the patient 10, in which case the data communication links 17 and 19 may be combined into a single data communication link. Alternatively, the user interface 40 may form part of the computer system 20, e.g. in the form of a peripheral device connected to the computer system 20 using a communication port or the like. In such a scenario, the actual physical condition of the patient 10 may be simulated by the processor arrangement 22 using the digital twin of the patient 10 using the provided user information in conjunction with the sensor data provided by the one or more sensors 12, 14, 16.

The one or more sensors 12, 14, 16 may be controlled by a controller 18 that is communicatively coupled to the computer system 20 through a control signal communication link 25. Although a single controller 18 is depicted that controls all sensors coupled to the patient 10, it should be understood that alternative arrangements in which at least some of the sensors have a dedicated controller 18 are of course equally feasible. Moreover, although the controller 18 is shown as a separate entity, it should be understood that this is by way of non-limiting example only, as it is equally feasible that the controller 18 forms part of a sensor it controls. Alternatively, the one or more sensors 12, 14, 16 may be manually controlled, e.g. by the patient 10. The processor arrangement 22 of the computer system 20 may control a mode of operation of such sensors through the controller 18, or by sending a control instruction to an electronic device in the possession of the patient 10 as will be explained in more detail below.

In order to avoid unnecessary use of the one or more sensors 12, 14, 16, each sensor may (initially) be operated in a mode of operation in which the sensor preserves energy, for example to extend the operational lifetime of a battery-powered sensor. For example, in case of a primary sensor for monitoring a particular physical condition of a patient 10, the sensor may be operated at a low data operating or sampling frequency, such as for example an operating frequency of 2 minutes per quarter of an hour for a heart rate monitoring sensor. At the same time, a sampling frequency of the sensor may be set to e.g. 30 to 240 samples per minute during an operating period of the sensor in order to facilitate measurement of the patient's heart beat frequency. In case of the presence of a secondary sensor for monitoring a particular physical state of a patient 10, e.g. a sensor verifying or supporting the primary sensor for monitoring a particular physical condition of a patient 10, such a secondary sensor may be switched off. More generally, the mode of operation of such a sensor may be controlled with the digital twin to strike a balance between the accuracy of the simulations of the patient's physical condition with the digital twin and the convenience of the patient 10, e.g. to preserve operational and battery lifetime of the sensor(s). However, this is not limited to reducing the need for the patient 10 to recharge or replace the batteries of the one or more sensors 12, 14, 16 but may also be intended to limit discomfort to the patent, e.g. by only performing ‘uncomfortable’ measurements such as blood pressure measurements when the digital twin considers such measurements necessary based on the simulated actual or future physical condition of the patient 10. Other non-limiting examples of effects of such sensor measurements potentially perceived as uncomfortable by the patient 10 include pressure changes, temperature variations, irritations to the skin, noise, visual changes (light), haptic changes (vibration) that may happen as a result of adapting the mode of operation of the sensor, as well as instructions or directions to the patient for triggering the patient to manually adjust a sensor setting, e.g. perform a measurement with a sensor.

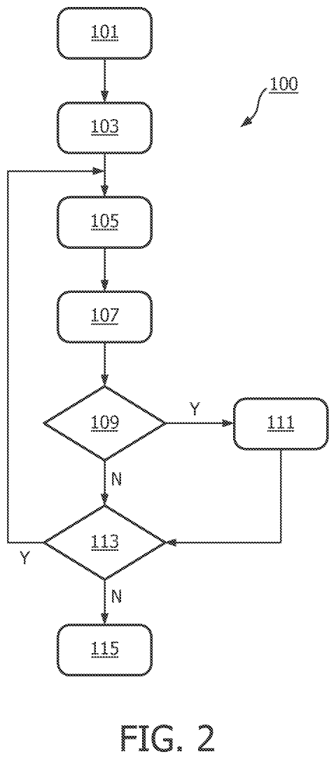

In accordance with a first set of embodiments of the present invention, the mode of operation of the one or more sensors 12, 14, 16 may be altered by the digital twin of the patient 10 as executed by the processor arrangement 22 of the computer system 20, as will be explained in further detail with the aid of FIG. 2, which depicts a flowchart of a method 100 of controlling the mode of operation of one or more sensors 12, 14, 16 of the patient 10 as implemented by the processor arrangement 22 of the computer system 20. The teachings of the method 100 will be explained in more detail using the arrangement of FIG. 1 as a non-limiting example without loss of generality. It will be understood by the skilled person that the embodiments of the method 100 of the present invention are not limited to a particular type of patient 10 and its associated digital twin and may be applied to any suitable type of patient 10 and its associated digital twin. Moreover, as will be demonstrated by some non-limiting examples of use cases of such a digital twin, the teachings of the present invention are not limited to a particular type of digital twin of the patient 10, as the digital twin may model any suitable part of the anatomy and/or physiology of the patient 10, such as a model of a lumen system of the patient 10, e.g. a model of part of the patient's cardiovascular, pulmonary or renal systems, a model of a blood chemistry system such as the blood glucose control system of the patient, a model of the pain experience of the patient, a fatigue model of the patient, and so on.

The method 100 commences in operation 101 after which the method 100 proceeds to operation 103 in which the digital representation (i.e. the digital twin) of the patient 10 is loaded onto the computer system 20, e.g. by retrieving the digital twin from the data storage device 30. Next, in operation 105 the computer system 20 receives the sensor data from the one or more sensors 12, 14, 16 over the data communication link 17, which sensor data as previously explained represents monitored physiological parameters of the patient 10, such as heart rate, blood pressure and respiration rate of a patient 10, from which the processor arrangement 22 may simulate an actual or future physical condition of the patient 10 using the digital twin, e.g. by updating one or more parameters of the digital twin with the received sensor data. The digital twin may implement a cardiovascular model of the patient 10 by way of non-limiting example. To this end, the sensor 12 may be a heart rate sensor, the sensor 14 may be a blood pressure sensor and the sensor 16 may be a respiration rate sensor. Of course, many other (medical) use cases of such digital twin technology will be immediately apparent to the skilled person as mentioned before. Operation 105 may further comprise receiving user information pertaining to a physical condition of the patient 10 by the computer system 20 through a user interface 40 as explained in further detail above.

In operation 107 of the method 100, the processor arrangement 22 develops the digital twin using the received sensor data in order to simulate the actual or future physical condition of the patient 10 and checks in operation 109 whether the actual or future physical condition of the patient 10 gives reason for concern, e.g. because it has significantly changed, for example by comparing the simulated actual or future physical condition of the patient 10 against one or more previously simulated or otherwise determined physical conditions of the patient 10 or by comparing it against some threshold or benchmark value. Such a significant change in the context of the present application may be a change in the physical condition of the patient 10 indicative of a loss of equilibrium in the patient 10. Such a comparison may in some embodiments be comparing an actual or predicted future value, e.g. a heart rate or the like, against a previously determined value or sequence of values, in which the comparison for example may be to check whether a trend in such a sequence of values is no longer followed by the actual or future value or whether such a trend predicts the patient's health becoming compromised, and so on. A difference between the actual or predicted future value and a previously determined value may be compared against a threshold to determine whether the change in the previously determined value is significant. Alternatively, such a threshold may define a boundary value of a safety window in which the physiological parameter should lie, such that exceeding such a threshold is indicative of the physiological parameter of the patient 10 indicating the patient 10 being in or approaching a potentially dangerous health condition.

If the simulated actual or future physical condition of the patient 10 does not give rise to any concerns, the method 100 may directly proceed to operation 113 in which the processor arrangement 22 determines if the monitoring of the patient 10 is to be continued, e.g. on the basis of a user input to this effect received through a user interface such as the user interface 40. If this is the case, the method 100 reverts back to operation 105; otherwise, the method 100 terminates in operation 100.

On the other hand, if the simulated actual or future physical condition of the patient 10 in operation 109 gives rise to concerns, e.g. because of a predicted change in this condition as explained above, the method 100 first proceeds to operation 111 in which the processor arrangement 22 generates a control instruction for one or more sensors 12, 14, 16 monitoring the patient 10 and provides this control instruction to the controller 18 of the appropriate sensor through the control instruction communication link 25, e.g. using the data communication module 24 in case of automatic control of the sensors 12, 14, 16 or to an electronic device (not shown) such as a smart phone, tablet computer or the like in the possession of the patient 10 in case of manual control of such sensors. The control instruction triggers the controller 18 to change the mode of operation of a particular sensor, or instructs the patient to manually change this mode of operation. Such a mode of operation for example may be a sampling frequency of that sensor, a number of samples taken by that sensor (e.g. to alter the accuracy of a parameter value derived from that number of samples), the change of a duration of a sample window deployed by that sensor, a data exchange frequency with the computer system 20 deployed by that sensor, the sensitivity of that sensor, the dynamic range of that sensor, the placement or location of that sensor, number of parameters measured with that sensor and so on. It should further be understood that such a mode of operation is not necessarily a static mode of operation, but may be a dynamic mode of operation instead, e.g. a monitoring function that changes as a function of time or the like.

Moreover, the mode of operation may relate to the switching on or addition of another sensor to the patient 10. For example, blood pressure (changes) may be estimated using PPG and acceleration sensors on the wrist, albeit at lower accuracy than with blood pressure monitors. Such monitoring may be done continuously in the absence of movement by the patient 10, as it is much more comfortable to the patient 10 than using a more accurate blood pressure cuff. However, when the digital twin determines that the measurements provided by the wrist-based sensors are indicative of a potential issue with the patient 10, the accuracy of the measurement of this parameter may need to be improved. In such case, either the patient 10 may already have been wearing an ambulatory blood pressure monitor which may then be inflated to measure blood pressure, or an instruction may be given to the patient 10 to add such a sensor (measurement), e.g. to measure blood pressure with such a monitor. In this manner, inconvenience to the patient 10 is reduced, because energy can be preserved by operating a sensor at a low sampling frequency as long as the physical condition of the patient 10 is in a state of equilibrium, or at least exhibits only insignificant changes as indicated by the sensor data produced by the one or more sensors 12, 14, 16 such that battery life, or more generally, operational life of such sensors is preserved for as long as possible or uncomfortable measurements to the patient are only performed when strictly necessary according to the digital twin. The justification of this approach is that the likelihood of sudden (potentially harmful) changes to the physical condition a stable patient 10 is negligible, such that a low sampling rate or frequency of such a sensor may be deployed with negligible risk to the patient 10. However, this risk can no longer be considered negligible once the digital twin simulation indicates that the physical condition of the patient 10 is no longer in a state of equilibrium such as for example indicated by a significant change in the patient's simulated physical condition as previously explained, as in such a scenario the risk of such sudden changes has increased. In such a scenario, it is therefore desirable to change the mode of operation of a targeted sensor in order to more accurately monitor further changes to the physical condition of the patient 10, i.e. in order to more accurately prevent undesired changes in the physical condition of the patient 10 from occurring.

This for example may further involve scheduling an appointment or procedure for a patient 10 exhibiting such changes to his or her physical (e.g. medical) condition, for example to prevent escalation of the physical condition to becoming potentially harmful or debilitating. For example, the original operational settings of the one or more sensors 12, 14, 16 may be defined by a medical practitioner or the digital twin based on the physical condition of the patient 10 at that particular time, after which changes to the operational settings of the sensors subsequently are being controlled by the digital twin of the patient based on sensor data (and user information such as user-reported symptoms). Hence, the operational lifetime and/or energy efficiency of the one or more sensors 12, 14, 16 is thereby optimized and/or measurements that are uncomfortable to the patient are minimized without increasing the risk of undesired changes to the patient 10.

In an embodiment, the control instruction issued by the processor arrangement 22 for the controller 18 of a targeted sensor 12, 14 and/or 16 specifying the change in mode of operation of the targeted sensor may include a specification of a duration of this mode of operation. For example, the control instruction may specify that the targeted sensor has to operate at a high sampling frequency for 1 hour, e.g. to monitor drug-induced changes to the patient's physiology. The controller 18 may be adapted to monitor an actual state of the battery of the targeted sensor and determine if the control instruction can be executed by the targeted sensor for the full duration specified in the control instruction. If this is indeed the case, the controller 18 may configure the targeted sensor in accordance with the received control instruction.

However, if the controller 18 determines that the remaining operational lifetime of the targeted sensor is insufficient to perform the control instruction for its full duration, the controller 18 may send a notification of the inability of the targeted sensor to comply with the control instruction to the computer system 20 over one of the aforementioned data links, which notification may further include an indication of the remaining operational lifetime of the targeted sensor, e.g. an indication of the actual duration at which the targeted sensor can operate at the specified mode of operation. Upon receiving this notification, the processor arrangement 22 may use the indication of the remaining operational lifetime of the targeted sensor to run one or more simulations with the patient's digital twin based on the operational lifetime limitations of the targeted sensor to decide how the control instruction is to be adjusted. For example, such simulations may include a first simulation at which the targeted sensor is operated at the desired sampling rate for a shorter duration as facilitated by the remaining operational lifetime of the targeted sensor and a second simulation at which the targeted sensor is operated at a reduced sampling rate for the desired duration as facilitated by the remaining operational lifetime of the targeted sensor. The simulation that provides the best match with the monitoring requirements of the patient 10 as predicted by the patient's digital twin is chosen to adjust the control instruction accordingly, after which the processor arrangement 22 issues the adjusted control instruction to the controller 18 of the targeted sensor, e.g. with the communication module 24.

The timing of the communication between the sensors 12, 14, 16 and their controller 18 (or the electronic device of the patient) and the computer system 20 may be handled in any suitable manner, as dictated by the needs of the digital twin. For example, the digital twin simulations and sensor adaptations can run in real time, and the sensors 12, 14, 16 can be adapted in (almost) real time. In other words, as the latest digital twin simulation results become available, these can be immediately used to generate the control instruction for one or more targeted sensors and communicate this instruction to the controller 18 of the targeted sensor(s).

Alternatively, the digital twin simulations may run at scheduled times, such that the modes of operation of targeted sensors could be also adapted at scheduled times if necessary. For example, running the digital simulations at point in time t=t0 and analysing the results, the processor arrangement 22 may decide that for the following time window [t1, t2] the mode of operation of a targeted sensor should be defined by a first mode of operation, whilst for a subsequent time window [t4, t5] the mode of operation of the targeted sensor should be defined by a second mode of operation. Moreover, the time for running the next digital twin simulation may be determined at t=t3 at the same time, i.e. based on the simulation output from t=t0, where t0<t1<t2<t3<t4<t5. Of course, where there is a decision to run another simulation at t=t3, the choice of the second mode of operation for the targeted sensor during time window [t4, t5] is preliminary at this stage, as this may be adjusted based on the results of the digital twin simulation to be performed at t=t3 using the sensor data collected during the time window [t1, t2]. The advantage of having preliminary indicators of what time window [t4, t5] and the second mode of operation of the targeted sensor should be from previous simulation runs, e.g. the simulation at t=t0, is that it assists in better planning and controlling available system resources (e.g. sensor memory and storage, battery lifetime, and so on). Such system resource monitoring may be used to strike a balance between simulation accuracy and system resource utilization.

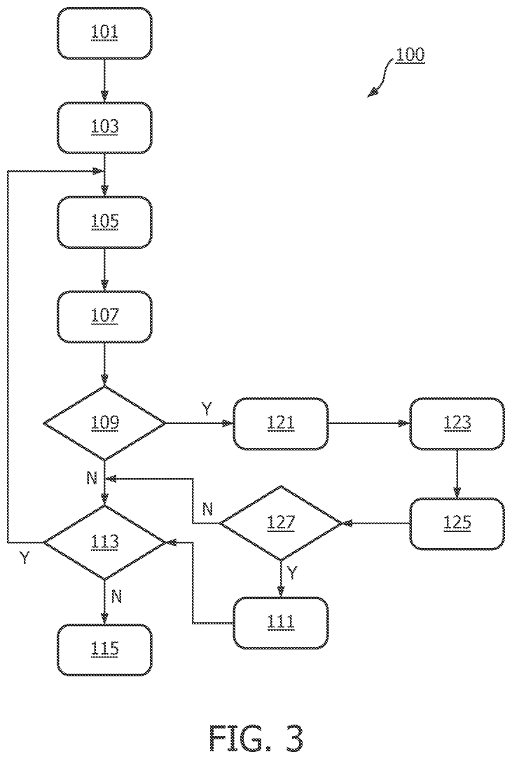

In a further refinement of the method 100 as depicted by the flowchart in FIG. 3, a physical condition of the patient 10 may be monitored with a first sensor (e.g. sensor 12) arranged to monitor a first physiological parameter of the patient 10 that is related to a second physiological parameter of the patient 10 and a second sensor (e.g. sensor 14) arranged to monitor the second physiological parameter. As a non-limiting example, the first sensor may monitor heart rate and the second sensor may monitor blood pressure or breathing parameters, which as is well-known are related as a change in heart rate is typically associated with a change in blood pressure and/or breathing rhythms or volume. In such a scenario, the method 100 may include an operation in which the processor arrangement 22 is arranged to simulate the actual or future physical condition of the patient 10 from sensor data provided by the first sensor in operation 107 as previously explained, and verify the simulated actual or future physical condition using sensor data provided by the second sensor. This for example may involve activating the second sensor by generating a control signal thereto and sending this control signal to the controller 18 of the second sensor or the electronic device in the possession of the patient 10, such that the controller 18 or the patient 10 may activate the second sensor accordingly. Of course, if the second sensor 18 is already active, the generation of such a control signal may be omitted.

To this end, the processor arrangement 22 may receive the sensor data of the second sensor in operation 123 (if not already received in operation 105) and simulate the actual or future physical condition of the patient 10 with its digital twin using the sensor data of the second sensor in operation 125 and in operation 127 evaluate this simulated actual or future physical condition, e.g. to determine if this simulated physical condition indicates a health concern for the patient 10. For example, if in operation 127 the actual or future physical condition of the patient 10 as simulated by the digital twin from the sensor data of the first sensor is verified (i.e. is substantially identical) with the actual or future physical condition as simulated by the digital twin from the sensor data of the second sensor, the control signal for controlling the mode of operation of the first sensor may be generated in operation 111. Otherwise, the method 100 proceeds to previously described operation 113.

Such verification of a simulated actual or future physical condition using a digital twin of a patient 10 may serve a number of purposes. For example, the first sensor may be a more energy efficient sensor than the second sensor, but the second sensor may provide more reliable sensor data to predict changes to an actual physical condition of the patient 10. In such a scenario, the first sensor may be preferably used to monitor the physical condition of the patient 10 for energy efficiency reasons whilst the physical condition is relatively stable, whereas upon predicted changes to the physical condition of the patient 10 with the digital twin based on the sensor data provided by the first sensor, such changes may be verified with the sensor data provided by the second sensor.

In another scenario, the simulated change to the physical condition of the patient 10 on the basis of the sensor data provided by the first sensor may predict a change in an operating parameter monitored by the second sensor. For example, a change in heart rate monitored with the first sensor may predict a change in blood pressure monitored with the second sensor. If the predicted change in the operating parameter monitored by the second sensor is not reproduced from the sensor data provided by the second sensor, this is an indication that the simulated actual physical condition of the patient 10 as based on the sensor data from the first sensor is likely to be inaccurate, such that the generation of the control signal in operation 111 to adjust mode of operation of the first sensor may be cancelled or made conditional of further information such as user information corroborating the predicted actual physical condition of the patient 10 based on the sensor data from the first sensor. To this end, the user may be invited to provide such user information, e.g. using a visible or audible message. Such a message for example may be produced on a device implementing the user interface 40, such as a smart phone, a tablet computer or the like. Of course, a dedicated user interface may be used instead.

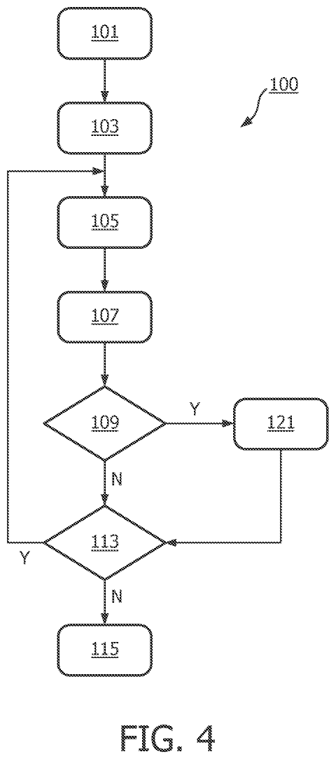

Another embodiment of the method 100 is depicted by the flowchart in FIG. 4. This embodiment differs from the embodiment depicted in FIG. 2 in that upon determination of an actual or future physical condition of the patient 10 giving cause for concern as simulated with its digital twin using sensor data from a first sensor monitoring a first operating parameter of the patient 10 the control signal generated by the processor arrangement 22 is an activation signal for a second sensor monitoring a second operating parameter of the patient 10 in operation 121. In this scenario, the second operating parameter is typically related to the first operating parameter such as for example a blood pressure and heart rate as previously explained.

The activation of the second sensor (and further sensors if required) may provide more detailed and/or accurate monitoring of the actual physical condition of the patient 10 upon disturbance of the equilibria of this physical condition, i.e. upon the physical condition being suspected to be in a state of flux. Consequently, further changes to the physical condition of the patient 10 are more elaborately monitored by monitoring a larger set of operating parameters pertaining to that physical condition such that any required future intervention can be more accurately forecasted, thereby reducing the risk of the patient 10 adopting an undesired physical condition before such intervention can take place. This for example is useful in scenarios where accurate monitoring of a particular physical condition is rather energy consuming, e.g. when using an optical sensor such as a PPG sensor, in which case such a physical condition is more approximately monitored using a more energy efficient sensor, such that only when the sensor data of the more energy efficient sensor indicates a significant change in the monitored physical condition of the patient 10 as simulated by the patient's digital twin on the processor arrangement 22 of the computer system 10, the processor arrangement 22 may produce a control instruction to activate the more accurate but more energy consuming sensor for monitoring changes to this physical condition.

In the above set of embodiments, the digital twin of the patient 10 as used by the processor arrangement 22 of the computer system 20 is used to perform a high-level simulation of an actual physical condition of the patient 10 based on the sensor data acquired by the one or more sensors 12, 14, 16 monitoring physiological parameters of the patient 10, optionally supplemented by user information such as symptoms or the like as previously explained. However, embodiments of the present invention are not limited to the instruction for controlling a mode of operation of at least one of such sensors based on the evaluation of the sensor data with the digital twin. In an alternative embodiment, such an instruction may be generated by the digital twin in response to a user request, e.g. as received through the user interface 40. For example, a healthcare professional may wish to run simulations using the digital twin to predict a future physical condition of the patient 10, e.g. to simulate the effect of certain treatment plans, which may be simulated by parameterizing the digital twin accordingly for instance. In such a scenario, the healthcare professional may obtain an accurate as possible starting point for such simulations, i.e. an accurate as possible model of actual physical condition of the patient 10. Therefore, the healthcare professional may request that the digital twin increases the accuracy of the simulation of the actual physical condition of the patient 10 by issuing the instruction for controlling a mode of operation of at least one of the sensors 12, 14, 16 such as to increase the accuracy and/or volume of the sensor data obtained from such sensors.

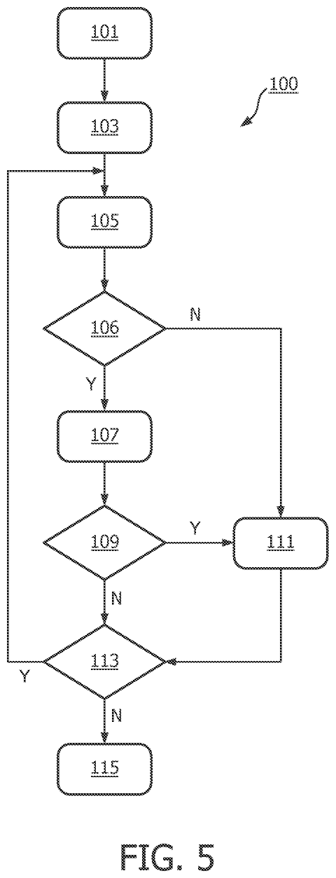

In another set of embodiments, the computer system 20 may utilize the digital twin to assess the relevance of the sensor data at a lower level, i.e. without necessarily utilizing the digital twin of the patient 10 to simulate an actual physical condition of the patient 10. This is depicted in the flowchart of the method 100 in FIG. 5, which differs from the previously described flowchart as depicted in FIG. 2 in that the method 100 comprises an additional evaluation operation 106 in which the processor arrangement 22 evaluates the relevance of the sensor data received in operation 105 to assess whether the sensor data can be reliably used to simulate the actual physical condition of the patient 10 in operation 107. Such an evaluation for example may assess whether the sensor data provided by a particular sensor is of low quality or is outdated.

Where the evaluation of the sensor data leads to the conclusion that the sensor data is sufficiently reliable, the method 100 may proceed to operation as previously described. However, if the evaluation of the sensor data leads to the conclusion that the sensor data is unreliable, e.g. outdated or of low quality, the method 100 may proceed to operation 111 in which the processor arrangement 22 generates a control instruction to rectify this issue. For example, in case of the evaluated sensor data being outdated, the processor arrangement 22 may generate a control instruction for the same sensor that generated this sensor data, e.g. a control instruction to reactivate this sensor or increase its sampling frequency. In another example, in case of the evaluated sensor data being of low quality or in case of missing sensor data, the processor arrangement 22 may generate a control instruction for another sensor that can acquire sensor data pertaining to the monitored physical condition of the patient 10 with the digital twin, as in such a scenario it may not be possible to rectify the unreliability of the sensor data with the sensor that generated this data.

The digital twin may further be utilized to generate the instruction to change the mode of operation of at least one of such sensors based on an evaluation of a hardware resource associated with the sensor operation or operation of the digital twin. For example, the digital twin may receive an operational life indication of the one or more sensors 12, 14, 16 with the sensor data produced by these sensors and generate this instruction in response to the operational life indication. For example, the digital twin may generate this instruction to reduce power consumption of the sensor targeted by the instruction where such a sensor has indicated that its battery depletion is imminent, if such a power consumption reducing change in the mode of operation of the sensor is clinically responsible. Alternatively or additionally, the digital twin may estimate the remaining lifetime of a sensor or one of its components and adjust the mode of operation of such a sensor accordingly. For example, operating a sensor for a prolonged period of time may result in a temperature increase of the sensor, which may reduce the lifetime of its components. This may be relevant in scenarios where the sensor network needs to be attached to and used by the patient 10 for long periods of time, as for instance is the case in patients suffering from Parkinson's disease who have to wear such sensors to receive guidance regarding medication dosing.

Such hardware resource evaluation in order to control the mode of operation at least some of the sensors monitoring the patient 10 may further include the determination of a remaining capacity of a data storage arrangement, e.g. a hard disk arrangement or memory, used by the virtual model (i.e. the digital twin) to store the sensor data and generate the instruction to change the mode of operation of a targeted sensor 12, 14, 16 in response to the determined remaining capacity.

At this point it is noted that the computer system 20 may further be adapted to store past communications between the digital twin and targeted sensors as well as past adaptations made to the mode of operation of a targeted sensor by the digital twin in addition to the simulation outputs of the digital twin. Such past communications and past adaptations may be used as a library or reference to assist the digital twin in determining how the mode of operation of a targeted sensor can be adapted, by searching such a library or reference to determine if a similar situation has previously occurred. Similarly, operating mode adaptations made by digital twins of different patients may be used to determine how the operating mode of the sensors for the patient 10 should be adapted for a predicted actual or future physical condition of the patient 10 with the digital twin.

The teachings of the present invention will now be explained in more detail by way of a number of examples of how digital twin technology may be used to monitor a physical condition of a patient 10. It should be understood that these examples are intended to explain rather than limit the teachings of the present invention, as it will be immediately apparent to the skilled person that many alternative use cases of such digital twin technology are readily available.

### EXAMPLE 1

### Patient-Specific Cardiovascular Monitoring

To monitor a patient 10 at risk of cardiovascular complications, a digital twin of a patient can be created that entails a patient-specific model of the cardiovascular system. A variety of such models are available, mostly classified into Finite Element (FE)-based and pressure-volume (PV) loop based, as for instance disclosed by B. W. Smith et al. in “Minimal Haemodynamic System Model Including Ventricular Interaction and Valve Dynamics” in Medical Engineering and Physics, 2004 (26), pages 131-139. Such models for example may be used to predict patient-specific hemodynamics, including blood pressure, heart rate, as well as ventricular load or coronary blood flow.

For such a patient, e.g. a patient suffering from heart failure, a digital twin may be created that predicts physiological parameters including intraventricular pressure, arterial blood pressure and cardiac output, and includes heart rate as one of its input parameters. To this end, the patient 10 may be fitted with a wearable sensor 12 containing a PPG and accelerometry sensor, which may be configured in an initial configuration to collect accelerometer-based parameters like number of steps taken by the patient 10 and activity type undertaken by the patient 10 continuously, and limit the PPG-based heart rate monitoring to 5 minutes each hour for the purpose of preserving the battery life of the sensor 12. The sensor data, e.g. the heart rate measurements may be used as input to the cardiovascular digital twin of the patient 10 to predict the actual health condition of the patient 10. This monitoring process is continued as long as the predicted health condition of the patient 10 as expressed by the monitored physiological parameters are within a defined acceptable range, e.g. as defined by a healthcare professional, and do not show any alarming trends.

However, as soon as a simulation result obtained with the virtual model (the patient's digital twin) gives reason for concern, e.g. a predicted cardiac output below a predefined threshold, the processor arrangement 22 generates the control instruction to change the mode of operation of the sensor 12 monitoring the patient 10. For example, the control instruction may cause the PPG sensor to increase the sampling frequency of the heart rate monitoring, such as to a continuous heart rate monitoring mode, in order to keep the digital twin input sensor data up-to-date. As a further option, the mode of operation of the wearable sensor 12 may be altered such as to extract additional parameters from the measured signals, including respiration rate, and blood pressure to obtain a more holistic approach to the monitoring of the patient's physical condition. This may involve the change of the mode of operation of other sensors worn by the patient 10 such as an ambulatory blood pressure monitor or an ECG patch to collect the aforementioned physiological parameters or other parameters pertaining to the physical state of the patient 10, as previously explained.

The additional sensor data may be used to directly monitor patient status and for example decide if an intervention is required and/or to verify the predictions made by the patient's digital twin. For example, if arterial blood pressure is also predicted by the cardiovascular digital twin and if this does not match the measured value, this may be an indication that the underlying patient-specific model parameters relating to the functioning of the patient's heart may have changed. This could indicate deterioration of a patient's condition and call for action, or be the result of a missed medication dosage of the patient. If the latter is suspected, a message may be sent to the patient 10 to verify this. Once the patient's physical condition has normalized (either automatically or by means of an intervention), the sensors of which their mode of operation has been adjusted may be returned to their initial (or another suitable) mode of operation.

### EXAMPLE 2

### Personalized Glucose Monitoring

Zeevi et al. in “Personal Nutrition by Prediction of Glycemic Responses”, Cell 2015 (5), Vol. 163, pages 1079-1094 have developed a model to predict an individual's glycemic response based on input parameters from a variety of sources such as microbiome, blood tests, questionnaires, anthropometrics, and the individual's food diary. This model can be used to create a personalized diet, for example to prevent the onset of diabetes, for a patient 10 at risk of this condition. Such a glycemic response model may be considered as a digital twin of the patient's glycemic response, and may be used to design a personalized diet to lower glycemic responses. The patient 10 may be given some devices for home monitoring, e.g. an activity tracker, an app to track food intake, a weight scale, and a finger prick device to measure blood glucose. Where the patient 10 is pre-diabetic or not at all diabetic, continuous glucose monitoring is unnecessary. Instead, the patient 10 may wear the activity tracker in accelerometer only mode to monitor activity (e.g. number of steps taken, activity type), and fill in a food diary. In addition, the patient 10 may check his blood glucose level from time to time with the finger prick device. This data can be used as input to the patient's digital twin (the glycemic response model) to predict the patient's glycemic response, and the measured blood glucose data can be used to check the accuracy of the model. The predictions obtained from the digital twin simulations using this data can be used to change the modes of operation of the patient monitoring devices (sensors) in a number of ways. For example, the predicted glycemic response, or a trend in a series of predicted glycemic responses may indicate an increased risk of diabetes to patient 10. To be able to give the patient 10 advice on lifestyle changes, more accurate input data may be required. As such, the heart rate measurements of the wearable sensor 12 may be switched on, which for example may result in more accurate estimations of energy expenditure, activity intensity, and cardio fitness of the patient 10. In addition, the patient may be encouraged to provide a body weight measurement on a daily basis, which measurements may be automatically or manually transferred to the computer system 20 hosting the patient's digital twin, and to measure blood glucose levels more often. These additional inputs enable more accurate personal glycemic predictions and improved personalized diet and activity guidance.

Where the predicted glycemic response is no longer in agreement with the measured response, this may indicate that some of the model parameters or inputs are incorrect or have become outdated. The aforementioned changes to the modes of operation of the various patient monitoring devices may also be made in this scenario. If such changes do not solve the issue, additional user input may be requested by means of the questionnaires used to build the glycemic response model as the patient's initial answers may be outdated, which if necessary may be supplemented by blood tests to determine blood glucose levels of the patient 10.

Once such issues have been resolved, e.g. the risk of diabetes has decreased to acceptable levels or the model predictions again accurately match measured blood glucose measurements, the adjusted modes of operation of affected patient monitoring devices (e.g. wearable sensors) may be changed to their initial (or another suitable) mode of operation.

### EXAMPLE 3

### Personalized Drug Delivery for Pain Management

In an intensive care unit or more generally for (post-)surgical patients, timely drug delivery is key for pain management. If medications are administered to the patient, e.g. orally or intravenously, it takes a certain amount of time before the medication wears off and a new dose needs to be delivered. A pain level predicting digital twin of the patient 10 that uses the input from different sensors worn or otherwise attached to the patient 10 can be used to predict the pain level experienced by the patient and consequently determine the best point in time to (re-)administer pain management drugs. However, as such pain experience typically is cyclical, it is not necessary to continuously monitor the patient 10, particularly in the early stages of such a pain cycle shortly after the administration of the pain management drug. More frequent monitoring and sensor data acquisition by the computer system 20 hosting the digital twin is necessary when the pain experienced by the patient 10 is predicted to increase again. In this manner, optimal drug delivery and sensor resource utilization can be achieved.

The above described embodiments of the method 100 executed by the processor arrangement 22 may be realized by computer readable program instructions embodied on a computer readable storage medium having, when executed on a processor arrangement 22 of a computer system 20, cause the processor arrangement 22 to implement any embodiment of the method 100. Any suitable computer readable storage medium may be used for this purpose, such as for example an optically readable medium such as a CD, DVD or Blu-Ray disc, a magnetically readable medium such as a hard disk, an electronic data storage device such as a memory stick or the like, and so on. The computer readable storage medium may be a medium that is accessible over a network such as the Internet, such that the computer readable program instructions may be accessed over the network. For example, the computer readable storage medium may be a network-attached storage device, a storage area network, cloud storage or the like. The computer readable storage medium may be an Internet-accessible service from which the computer readable program instructions may be obtained. In an embodiment, the computer system 20 is adapted to retrieve the computer readable program instructions from such a computer readable storage medium and to create a new computer readable storage medium by storing the retrieved computer readable program instructions in a data storage arrangement 30 accessible to the computer system 20, e.g. in a memory device or the like forming part of the computer system 20.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. The word “comprising” does not exclude the presence of elements or steps other than those listed in a claim. The word “a” or “an” preceding an element does not exclude the presence of a plurality of such elements. The invention can be implemented by means of hardware comprising several distinct elements. In the device claim enumerating several means, several of these means can be embodied by one and the same item of hardware. The mere fact that certain measures are recited in mutually different dependent claims does not indicate that a combination of these measures cannot be used to advantage.

1. A computer system comprising:
a processor arrangement communicatively coupled to a data storage arrangement storing a virtual model of a patient, said virtual model comprising at least one of a digital representation of at least a part of the anatomy of the patient and a physiological model of a bodily process of the patient; and
a communication module communicatively coupled to said processor arrangement and arranged to receive sensor data from one or more sensors arranged to monitor said patient, wherein the processor arrangement is arranged to:
retrieve said virtual model from the data storage arrangement;
receive said sensor data from the communication module;
evaluate said sensor data with said virtual model;
generate an instruction for altering a mode of operation of at least one sensor of the one or more sensors in response to said evaluation or in response to a user request; and
transmit said instruction to the at least one sensor or to a device for invoking control of said at least one sensor with the communication module.

2. The computer system of claim 1, wherein the processor arrangement is arranged to:
evaluate said sensor data with said virtual model by simulating an actual or future physical condition of said patient by developing said digital representation based on the received sensor data;
and generate said instruction based on said simulated actual or future physical condition.

3. The computer system of claim 2, wherein the processor arrangement is arranged to simulate the actual or future physical condition of said patient by developing said digital representation based on the received sensor data and received user information indicative of said actual physical condition.

4. The computer system of claim 2, wherein the one or more sensors include a first sensor arranged to monitor a first physiological parameter of the patient that is related to a second physiological parameter of the patient and a second sensor arranged to monitor said second operating parameter, and wherein the processor arrangement is arranged to:
simulate the actual or future physical condition from sensor data provided by the first sensor;
verify the simulated actual or future physical condition from sensor data provided by the second sensor; and
generate the instruction based on said simulated actual or future physical condition if said simulated actual or future physical condition has successfully been verified.

5. The computer system of claim 4, wherein the processor arrangement is further arranged to generate a further instruction for activating the second sensor and transmitting the further instruction to the second sensor with the communication module based on said simulated actual or future physical condition.

6. The computer system of claim 2, wherein:
the one or more sensors include a first sensor arranged to monitor a first physiological parameter of the patient that is related to a second physiological parameter of the patient and a second sensor arranged to monitor said second physiological parameter; and
the processor arrangement is arranged to generate a further instruction for activating the second sensor and transmitting the further instruction to the second sensor with the communication module based on said simulated actual or future physical condition.

7. The computer system of claim 1, wherein the processor arrangement is arranged to evaluate said sensor data with said virtual model by at least one of:
determining a quality indicator of said sensor data and generating said instruction if said quality indicator is below a defined threshold; and
receiving an operational life indication of the one or more sensors with said sensor data and generating said instruction in response to said operational life indication.

8. The computer system of claim 1, wherein the processor arrangement is arranged to determine a remaining capacity of a data storage arrangement used by the virtual model to store the sensor data and generate said instruction in response to said determined remaining capacity.

9. The computer system of claim 1, wherein the processor arrangement is further arranged to adjust said instruction and transmit said adjusted instruction to the at least one sensor with the communication module in response to an indication of an inability to comply with the original instruction from the at least one sensor.

10. A method of controlling one or more sensors arranged to monitor a patient with a computer system comprising:
a processor arrangement communicatively coupled to a data storage arrangement storing a virtual model of the patient, said virtual model comprising at least one of a digital representation of at least a part of the anatomy of the patient and a physiological model of a bodily process of the patient; and
a communication module communicatively coupled to said processor arrangement and arranged to receive sensor data from one or more sensors arranged to monitor said patient, the method comprising, with said processor arrangement:
retrieving said virtual model from the data storage arrangement;
receiving said sensor data from the communication module;
evaluating said sensor data with said virtual model;
generating an instruction for altering a mode of operation of at least one sensor of the one or more sensors in response to said evaluation or in response to a user request; and
transmitting said instruction to the at least one sensor or to a device for invoking control of said at least one sensor with the communication module.

11. The method of claim 10, wherein evaluating said sensor data with said virtual model comprises:
simulating an actual or future physical condition of said patient by developing said digital representation based on the received sensor data; and
generating said instruction based on the simulated actual or future physical condition.

12. The method of claim 11, wherein the one or more sensors include a first sensor arranged to monitor a first physiological parameter of the patient that is related to a second physiological parameter of the patient and a second sensor arranged to monitor said second operating parameter, and wherein the method comprises:
simulating the actual or future physical condition from sensor data provided by the first sensor;
verifying the simulated actual or future physical condition from sensor data provided by the second sensor; and
generating the instruction based on the simulated actual or future physical condition if said simulated actual or future physical condition has successfully been verified, the method optionally further comprising generating a further instruction for activating the second sensor with the processor arrangement and transmitting the further instruction to the second sensor with the communication module based on the simulated actual or future physical condition.

13. The method of claim 11, wherein the one or more sensors include a first sensor arranged to monitor a first physiological parameter of the patient that is related to a second physiological parameter of the patient and a second sensor arranged to monitor said second physiological parameter; and the method further comprising generating a further instruction for activating the second sensor with the processor arrangement and transmitting the further instruction to the second sensor with the communication module based on the simulated actual or future physical condition.

14. The method of claim 10, further comprising evaluating said sensor data with said virtual model by at least one of:
determining a quality indicator of said sensor data and generating said instruction if said quality indicator is below a defined threshold;
receiving an operating life indication of the one or more sensors with said sensor data and generating said instruction in response to said operating life indication; and
determining a remaining capacity of a data storage arrangement used by the virtual model to store the sensor data and generating said instruction in response to said determined remaining capacity.

15. A computer program product for a computer system comprising a processor arrangement communicatively coupled to a data storage arrangement storing a virtual model of a patient, said virtual model comprising at least one of a digital representation of at least a part of the anatomy of the patient and a physiological model of a bodily process of the patient; and
a communication module communicatively coupled to said processor arrangement and arranged to receive sensor data from said one or more sensors;
the computer program product comprising a computer readable storage medium having computer readable program instructions embodied therewith for, when executed on the processor arrangement, cause the processor arrangement to implement the method of claim 10.
